(19) United States
(12) Patent Application Publication
(10) Pub. No.: US 2025/0205480 A1
(43) Pub. Date: Jun. 26, 2025

(54) BEAUTY APPARATUS

(71) Applicant: XIAMEN SOLEX HIGH-TECH INDUSTRIES CO., LTD., Fujian (CN)

(72) Inventors: Caigui Lin, Fujian (CN); Guihua Yan, Fujian (CN); Tingke Liu, Fujian (CN); Mingfu Zhang, Fujian (CN)

(73) Assignee: XIAMEN SOLEX HIGH-TECH INDUSTRIES CO., LTD., Fujian (CN)

(21) Appl. No.: 18/842,637

(22) PCT Filed: Mar. 22, 2023
(86) PCT No.: PCT/CN2023/083133

§ 371 Date: Aug. 29, 2024

(30) Foreign Application Priority Data
Apr. 6, 2022 (CN) 202210354492.9

(51) Int. Cl. A61N 1/32; A61N 1/40

(52) U.S. Cl. CPC A61N 1/328; A61N 1/40

(57) ABSTRACT

A beauty apparatus includes a housing, an electrode assembly, and a lift driving member. The electrode assembly includes a first electrode assembly and a second electrode assembly. The lift driving member is located in the housing and configured to drive the second electrode assembly to move between a first position and a second position. When the second electrode assembly is located in the first position, the effective area of the electrode assembly capable of acting on the human skin surface is S1. When the second electrode assembly is located in the second position, the effective area of the electrode assembly capable of acting on the human skin surface is S2. S1>S2. The preceding beauty apparatus helps beautify a small area of skin.

This disclosure claims priority to Chinese Patent Application No. 202210354492.9, entitled “BEAUTY APPARATUS”, filed Apr. 6, 2022, the disclosure of which is incorporated herein by reference in its entirety.

### TECHNICAL FIELD

The present disclosure relates to a personal care apparatus and, in particular, to a beauty apparatus.

### BACKGROUND

In recent years, people have raised their demand for a higher quality of material and spiritual life with the rapid development of China's economy, the progress of science and technology, and the increasing improvement of people's living standards. People's pursuit of beauty is no longer satisfied with the decorative beauty outside their bodies, and more attention is paid to the natural beauty displayed by their bodies. Therefore, the beauty industry has developed rapidly, and beauty apparatuses under high and new technology have developed accordingly. Miniature and portable beauty apparatuses have gradually entered people's lives. Radio frequency beauty apparatuses or micro-current beauty apparatuses become relatively common beauty apparatuses.

A radio frequency beauty apparatus or a micro-current beauty apparatus needs to contact the skin directly and implements various beauty and skin care effects through the current of an electrode acting on the skin. However, the electrode area in an existing radio frequency beauty apparatus or micro-current beauty apparatus is relatively large, which is suitable for acting on a large piece of skin but difficult to apply to the skin of an eye or a lip. Therefore, existing radio frequency beauty apparatuses or micro-current beauty apparatuses have certain limitations.

### SUMMARY

The main technical problem to be solved by the present disclosure is to provide a beauty apparatus, which is convenient for beautifying a small area of skin.

According to an aspect of the present disclosure, the present disclosure provides a beauty apparatus including a housing, an electrode assembly, and a lift driving member.

The electrode assembly includes a first electrode assembly and a second electrode assembly. The lift driving member is located in the housing and configured to drive the second electrode assembly to move between a first position and a second position. When the second electrode assembly is located in the first position, an effective area of the electrode assembly capable of acting on a human skin surface is S1. When the second electrode assembly is located in the second position, the effective area of the electrode assembly capable of acting on the human skin surface is S2. S1>S2.

According to an embodiment of the present disclosure, the second electrode assembly includes a second electrode tip and a lifting rod. The lifting rod is securely connected to the second electrode tip. Conductive pieces are respectively disposed on two sides of the lifting rod in a radial direction of the lifting rod.

The first electrode assembly is provided with a mount for mounting a first electrode tip. The mount is provided with an avoidance cavity for accommodating the second electrode assembly. An inner wall of the avoidance cavity has contact points respectively electrically connected to the conductive pieces.

According to an embodiment of the present disclosure, the lift driving member drives, based on manpower or electricity, the lifting rod to move in an axial direction of the lifting rod.

According to an embodiment of the present disclosure, the lift driving member includes a gear motor and a rotation sleeve. The gear motor is configured to drive the rotation sleeve to rotate. The rotation sleeve is connected to the lifting rod through a conversion member. The conversion member is configured to convert a rotation of the rotation sleeve into a movement of the lifting rod in the axial direction of the lifting rod.

According to an embodiment of the present disclosure, the lift driving member includes a rotary knob and a rotation sleeve. The rotary knob is configured to drive the rotation sleeve to rotate. The rotation sleeve is connected to the lifting rod through a conversion member. The conversion member is configured to convert a rotation of the rotation sleeve into a movement of the lifting rod in the axial direction of the lifting rod.

According to an embodiment of the present disclosure, the rotary knob is disposed on a bottom of the housing in the axial direction of the housing. The rotary knob is securely connected to the rotation sleeve through a connection rod.

According to an embodiment of the present disclosure, the rotary knob is disposed on a top of the housing in the axial direction of the housing. The rotary knob is secured to the rotation sleeve by inserting.

According to an embodiment of the present disclosure, the rotary knob is the mount.

According to an embodiment of the present disclosure, the rotation sleeve is provided with an avoidance channel for the lifting rod to pass through. The conversion member includes a spiral guide rail and a guide block, where the spiral guide rail is disposed on an inner wall of the avoidance channel, and the guide block is disposed on the lifting rod. The guide block is disposed in the spiral guide rail.

According to an embodiment of the present disclosure, the lift driving member includes a push rod motor. A telescopic end of the push rod motor is cooperatively linked to the lifting rod.

According to an embodiment of the present disclosure, the lift driving member includes a push button securely connected to the lifting rod. The housing is provided with a sliding recess for the push button to translate. The sliding recess extends in the axial direction of the lifting rod. One end of the push button is securely connected to the lifting end. Another end of the push button is exposed outside the sliding recess to form an operation end.

According to an embodiment of the present disclosure, the lift driving member includes an elastic member and a release member. When the second electrode assembly is located in the first position, the elastic member retracts in the axial direction of the lifting rod and is in an energy storage state. The release member is configured to unlock the second electrode assembly from the first position.

According to an embodiment of the present disclosure, a release member includes a button. The button is connected to a swing rod. An engagement block for engaging with and limiting a swing end of the swing rod is disposed on the lifting rod. When the second electrode assembly is located in the first position, the swing end engages with and limits the engagement block so as to secure the second electrode assembly in the first position. When the button is pressed, the engagement block disengages from the swing end and stops limiting the swing end.

According to an embodiment of the present disclosure, an electrode in the first electrode assembly and an electrode in the second electrode assembly are each a micro-current positive or negative contact point, or are each a radio frequency electrode.

According to an embodiment of the present disclosure, in a process of the second electrode assembly moving from the first position to the second position, the second electrode tip in the second electrode assembly protrudes outward or recesses inward relative to the first electrode tip in the first electrode assembly.

According to an embodiment of the present disclosure, the second electrode assembly and the first electrode assembly are arranged concentrically from inside to outside in the radial direction of the first electrode assembly and the second electrode assembly.

Compared with the existing art, technical solutions of the present disclosure have the beneficial effects below.

The present disclosure provides a beauty apparatus. The second electrode assembly is driven through the lift driving member to move between the first position and the second position, thereby part of an electrode tip has the lifting function. In this case, when a small area of skin, such as an eye or a lip, needs to be beautified, the second electrode assembly can be lifted or retracted so as to reduce the effective area of the electrode tip acting on the human skin surface, thereby adapting to the small area of skin for use.

### BRIEF DESCRIPTION OF DRAWINGS

The above and other features and advantages of the present disclosure become more apparent by describing the example embodiments thereof in detail with reference to the drawings.

FIG. 1 is a perspective view when a second electrode assembly is in a first position in embodiment one of the present disclosure.

FIG. 2 is a perspective view when the second electrode assembly is in a second position in embodiment one of the present disclosure.

FIG. 3 is a section view when the second electrode assembly is in the first position in embodiment one of the present disclosure.

FIG. 4 is a section view when the second electrode assembly is in the second position in embodiment one of the present disclosure.

FIG. 5 is an exploded view in embodiment one of the present disclosure.

FIG. 6 is a partial section view when the second electrode assembly is in the first position in embodiment one of the present disclosure.

FIG. 7 is a partial section view when the second electrode assembly is in the second position in embodiment one of the present disclosure.

FIG. 8 is an exploded view in embodiment two of the present disclosure.

FIG. 9 is a partial section view when the second electrode assembly is in the first position in embodiment two of the present disclosure.

FIG. 10 is a partial section view when the second electrode assembly is in the second position in embodiment two of the present disclosure.

FIG. 11 is an exploded view in embodiment three of the present disclosure.

FIG. 12 is a partial section view when the second electrode assembly is in the first position in embodiment three of the present disclosure.

FIG. 13 is a partial section view when the second electrode assembly is in the second position in embodiment three of the present disclosure.

FIG. 14 is an exploded view in embodiment four of the present disclosure.

FIG. 15 is a partial section view when the second electrode assembly is in the first position in embodiment four of the present disclosure.

FIG. 16 is a partial section view when the second electrode assembly is in the second position in embodiment four of the present disclosure.

FIG. 17 is an exploded view in embodiment five of the present disclosure.

FIG. 18 is a section view when the second electrode assembly is in the first position in embodiment five of the present disclosure.

FIG. 19 is a section view when the second electrode assembly is in the second position in embodiment five of the present disclosure.

FIG. 20 is an exploded view in embodiment six of the present disclosure.

FIG. 21 is a section view when the second electrode assembly is in the first position in embodiment six of the present disclosure.

FIG. 22 is a section view when the second electrode assembly is in the second position in embodiment six of the present disclosure.

### DETAILED DESCRIPTION

Technical solutions in embodiments of the present disclosure are described clearly and completely hereinafter in conjunction with the accompanying drawings in embodiments of the present disclosure. Apparently, the embodiments described below are part, not all, of embodiments of the present disclosure. Based on embodiments of the present disclosure, all other embodiments obtained by those of ordinary skill in the art without creative work are within the scope of the present disclosure.

In the description of the present disclosure, it is to be noted that the orientational or positional relationships indicated by terms “above”, “below”, “inside”, “outside”, “top”, “bottom” and the like are based on the orientational or positional relationships illustrated in the drawings, which are for the mere purpose of facilitating and simplifying the description of the present disclosure, and these relationships do not indicate or imply that the device or element referred to has a specific orientation and is constructed and operated in a specific orientation, and thus it is not to be construed as limiting the present disclosure. In addition, terms such as “first” and “second” are used merely for the purpose of description and are not to be construed as indicating or implying relative importance.

In the description of the present disclosure, it is to be noted that unless otherwise expressly specified and limited, terms like “mounted”, “configured”, “sleeved”, or “connected” should be construed in a broad sense as. For example, the term “connected” may be connected in a wall-mounted manner, detachably connected or integrally connected; mechanically connected or electrically connected; directly connected to each other or indirectly connected to each other via an intermediary; or intraconnected between two components. For those of ordinary skill in the art, specific meanings of the preceding terms in the present disclosure may be understood based on specific situations.

### Embodiment One

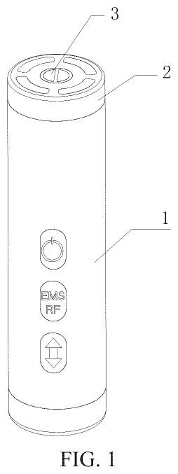

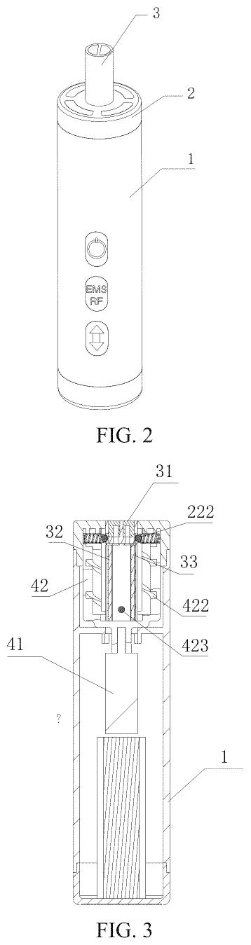

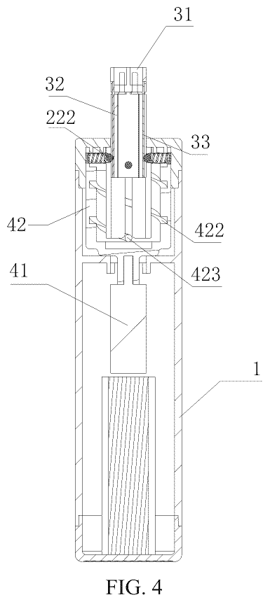

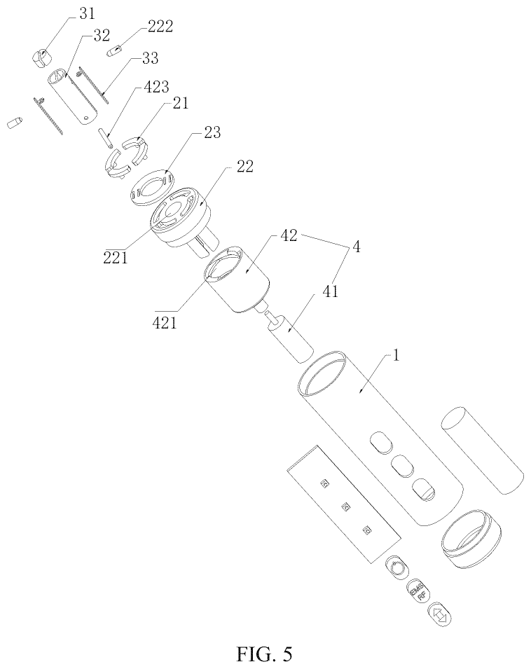

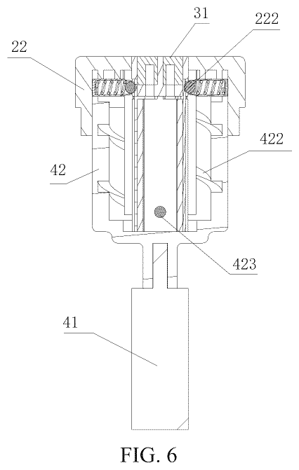

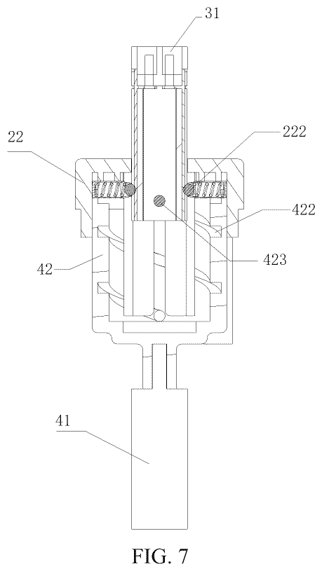

Referring to FIGS. 1 to 7, this embodiment provides a beauty apparatus. The beauty apparatus includes a housing 1, an electrode assembly, and a lift driving member 4. The electrode assembly includes a first electrode assembly 2 and a second electrode assembly 3. The lift driving member 4 is located in the housing 1 and configured to drive the second electrode assembly 2 to move between a first position and a second position. When the second electrode assembly 3 is located in the first position, a second electrode tip 31 of the second electrode assembly 3 and a first electrode tip 21 of the first electrode assembly 2 are located in the same plane. When the second electrode assembly 3 is located in the second position, the second electrode tip 31 of the second electrode assembly 3 protrudes outside the first electrode tip 21 of the first electrode assembly 2. In this case, when the second electrode assembly 3 is located in the first position, the effective area of the electrode assembly capable of acting on the human skin surface is S1; and when the second electrode assembly 3 is located in the second position, the effective area of the electrode assembly capable of acting on the human skin surface is S2. S1>S2.

In this case, when a beauty treatment is performed, the position of the second electrode assembly 3 can be selected according to a region to be beautified, thereby implementing a change in the effective area of an electrode tip. For example, when a region with a relatively large area of skin, such as a forehead or a cheek, is beautified, a user can operate the second electrode assembly 3 to be located in the first position so that the effective area of an electrode tip is a sum of the area of the electrode tip in the first electrode assembly 2 and the area of the electrode tip in the second electrode assembly 3. When a region with a relatively small area of skin, such as a lip or an eye, is beautified, the user can operate the second electrode assembly 3 to be located in the second position so that the effective area of an electrode tip is merely the area of the electrode tip in the second electrode assembly 3, facilitating the operation in a narrow region.

It is to be noted that by way of example, the second electrode tip 31 extends outward and protrudes outside the first electrode tip 21 in this embodiment. The second electrode tip 31 may also recess inward so that the first electrode tip 21 protrudes outward, thereby reducing the area of an electrode tip and facilitating the operation in a narrow region. Therefore, as long as two electrode tips are not in the same plane through the relative movement of the electrode tips, the concept of this embodiment can be realized, which belongs to a simple replacement of this embodiment. Moreover, in this embodiment, when the second electrode assembly 3 is in the first position, the first electrode tip 21 and the second electrode tip 31 are located in the same plane. As a simple replacement, the two electrode tips may also be located on one arc surface. Alternatively, a slight height difference exists between the two electrode tips, and the height difference does not affect the joint action of the first electrode tip 21 and the second electrode tip 31 on human skin. These all belong to simple replacements of this embodiment.

In this embodiment, the second electrode assembly 3 has the lifting function, and the second electrode assembly 3 needs to have the basic functions of an electrode assembly in either the first position or the second position. In this case, the electrical connection between the second electrode assembly 3 and a circuit board needs to be maintained. Accordingly, the second electrode assembly 3 includes the second electrode tip 31 and a lifting rod 32 securely connected to the second electrode tip 31. Conductive pieces 33 are disposed on two sides of the lifting rod 33 in a radial direction of the lifting rod 33. One end of the conductive piece 33 extends into the lifting rod 32 and is electrically connected to the second electrode tip 31 disposed on an end portion of the lifting rod 32.

The first electrode assembly 2 is provided with a mount 22 for mounting the first electrode tip 21. The mount 22 is provided with an avoidance cavity 221 for accommodating the second electrode assembly. 3 An inner wall of the avoidance cavity 221 has a contact point 222 electrically connected to the conductive piece 33. The circuit board 23 and the first electrode tip 21 are mounted on the mount 22. In this case, the conductive piece 33 can be electrically connected to the second electrode tip 31 and the circuit board 23. The conductive piece 33 extends in the axial direction of the lifting rod 32 so that even if the second electrode tip 31 moves relative to the contact point 222, other positions on the conductive piece 33 can still be electrically connected to the contact point 222, guaranteeing the stability of the electrical connection of the second electrode tip 32 in the movement process. In this embodiment, the first electrode tip 21 and the second electrode tip 31 may be each a radio frequency tip, or be each a micro-current positive or negative contact point. Apart from circuits corresponding to radio frequency tips or micro-current positive and negative contact points, an LED chip and a corresponding driver circuit may also be disposed on the circuit board 23, thereby implementing the effect of phototherapy.

In this embodiment, the lift driving member drives, based on electricity, the lifting rod 32 to move. The lift driving member 4 includes a gear motor 41 and a rotation sleeve 42. The gear motor 41 is configured to drive the rotation sleeve 42 to rotate. The rotation sleeve 42 is connected to the lifting rod 32 through a conversion member. The conversion member is configured to convert the rotation of the rotation sleeve 42 into the movement of the lifting rod 32 in the axial direction of the lifting rod 32.

To implement the preceding converting effect, the rotation sleeve 42 is provided with an avoidance channel 421 for the lifting rod 32 to pass through. The conversion member includes a spiral guide rail 422 and a guide block, where the spiral guide rail 422 is disposed on an inner wall of the avoidance channel 421, and the guide block is disposed on the lifting rod 32. The guide block is disposed in the spiral guide rail 422. In this embodiment, a mounting hole is disposed on each of two sidewalls of the lifting rod 32. One horizontal rod 423 passes through mounting holes, and two ends of the horizontal rod 423 are exposed outside outer walls of the lifting rod 32 to form the guide block.

In this embodiment, the second electrode assembly and the first electrode assembly are arranged concentrically from inside to outside in the radial direction of the first electrode assembly and the second electrode assembly.

### Embodiment Two

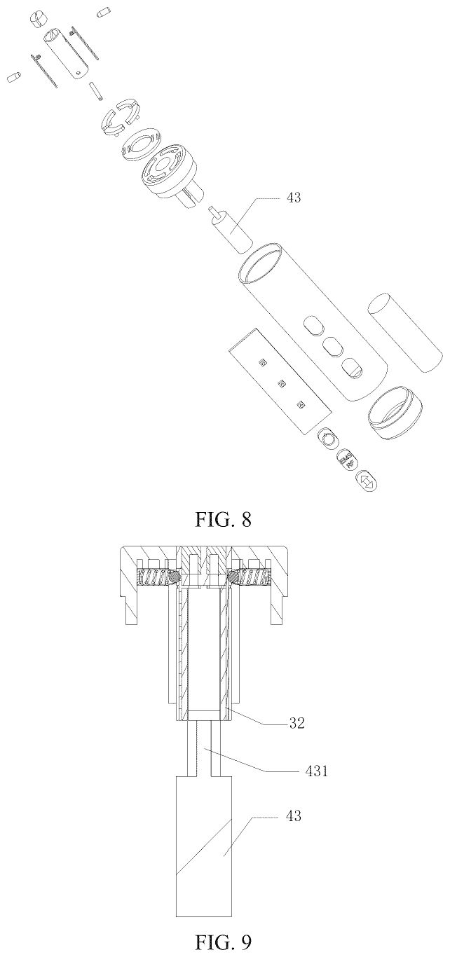

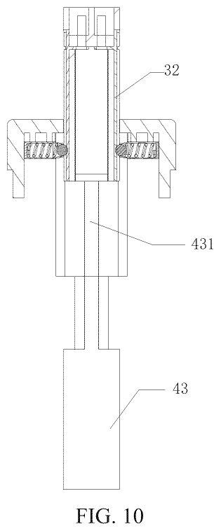

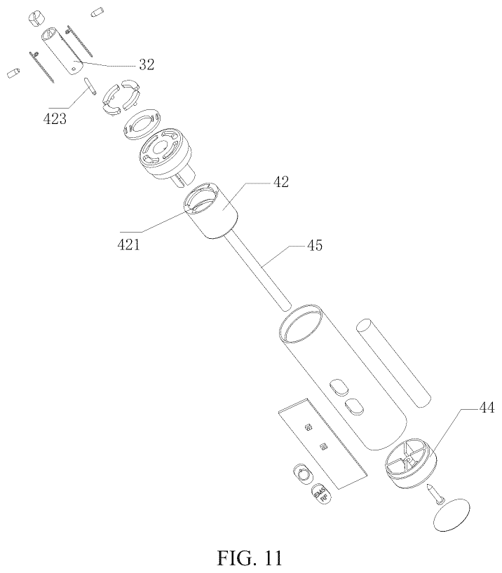

Referring to FIGS. 8 to 10, the difference between this embodiment and embodiment one lies in that a push rod motor is used in this embodiment. In this case, the rotation sleeve in embodiment one can be eliminated. The lifting rod 32 is driven directly through a telescopic end 431 of the push rod motor 43 to move.

### Embodiment Three

Embodiment one and embodiment two are both solutions in which a motor drives the lifting rod 32. To simplify the structure and reduce the cost, manpower is used in this embodiment to drive the lifting rod 32 to lift.

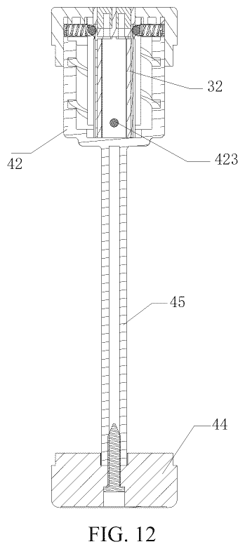

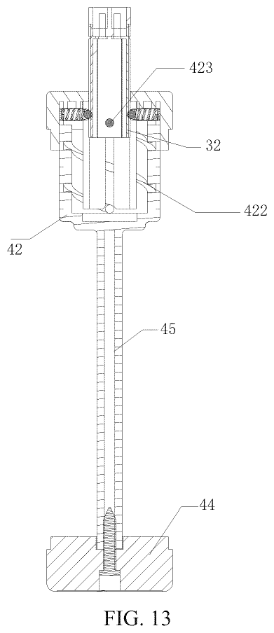

Accordingly, referring to FIGS. 11 to 13, the lift driving member 4 includes a rotary knob 44 and a rotation sleeve 42. The rotary knob 44 is configured to drive the rotation sleeve 42 to rotate. The rotation sleeve 42 is connected to the lifting rod 32 through a conversion member. The conversion member is configured to convert the rotation of the rotation sleeve 42 into the movement of the lifting rod 32 in the axial direction of the lifting rod 32

To implement the preceding converting effect, the rotation sleeve 42 is provided with an avoidance channel 421 for the lifting rod 32 to pass through. The conversion member includes a spiral guide rail 422 and a guide block, where the spiral guide rail 422 is disposed on an inner wall of the avoidance channel 421, and the guide block is disposed on the lifting rod 32. The guide block is disposed in the spiral guide rail 422. In this embodiment, a mounting hole is disposed on each of two sidewalls of the lifting rod 32. One horizontal rod 423 passes through mounting holes, and two ends of the horizontal rod 423 are exposed outside outer walls of the lifting rod 32 to form the guide block.

In this embodiment, the rotary knob 44 is disposed on the bottom of the housing 1 in the axial direction. The rotary knob 44 is securely connected to the rotation sleeve 42 through a connection rod 45. The user turns the rotary knob 44 to drive the rotation sleeve 42 through the connection rod 45 to rotate. The rest is the same as embodiment one and is not repeated.

### Embodiment Four

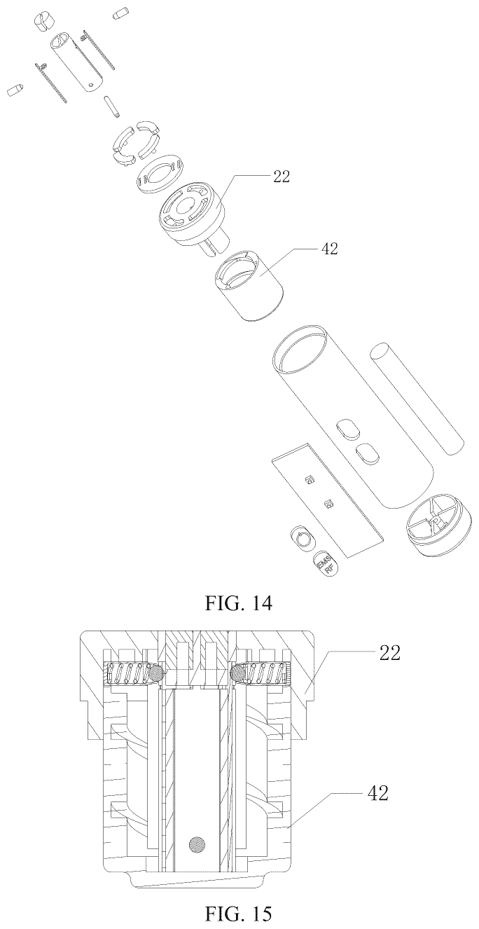

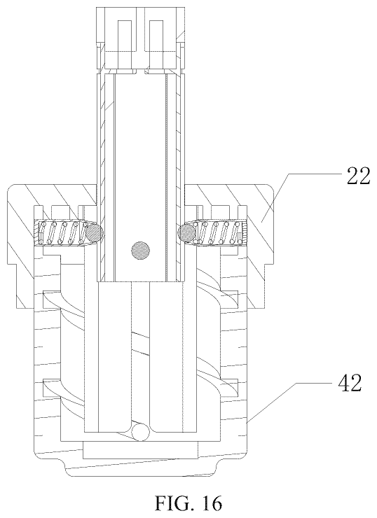

Referring to FIGS. 14 to 16, the difference between this embodiment and embodiment three lies in that the rotary knob is disposed on the top of the housing 1 in the axial direction of the housing and that the rotary knob is secured to the rotation sleeve by inserting. In this case, the mount 22 of the first electrode assembly 2 can be used as the rotary knob. The rotation sleeve 42 is inserted into the mount 22 so that the rotation sleeve 42 is driven to rotate when the mount 22 is operated to rotate. The arrangement in which the mount 22 serves as the rotary knob 44 requires no additional rotary knob, further simplifying the structure.

### Embodiment Five

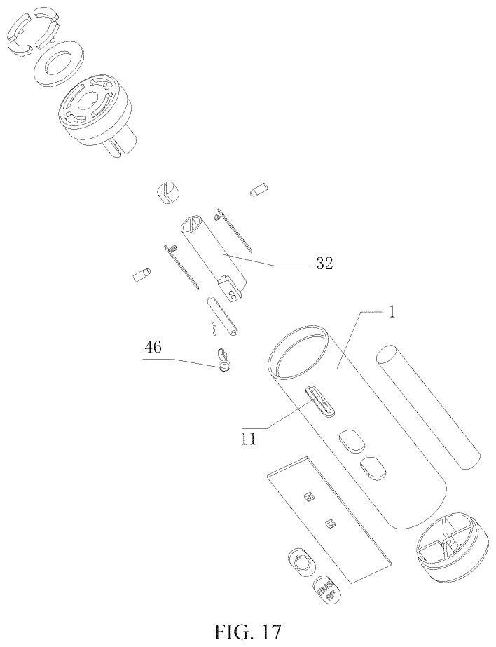

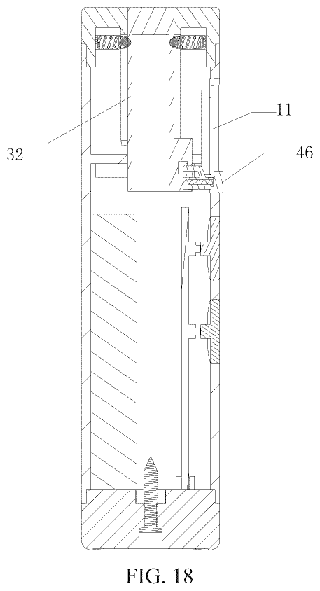

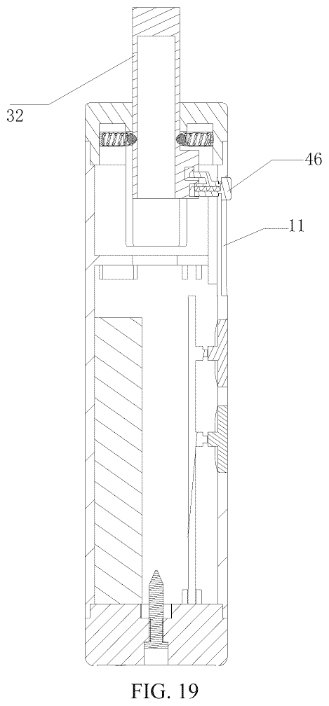

Referring to FIGS. 17 to 19, embodiment three and embodiment four are solutions in which the rotary knob is driven based on manpower to rotate and then the lifting rod 32 is driven to move through the release member. In this embodiment, the lifting rod 32 is directly driven based on manpower to move without conversion.

Accordingly, the lift driving member 4 includes a push button 46 securely connected to the lifting rod 32. In the axial direction of the lifting rod 32, the housing 1 is provided with a sliding recess 11 for the push button 46 to translate. One end of the push button 46 is securely connected to the lifting end 32. The other end of the push button 46 is exposed outside the sliding recess 11 to form an operation end. The user needs to push the operation end upward or downward so as to drive the lifting rod 32 to move upward or downward, thereby enabling the second electrode assembly 3 to move between the first position and the second position.

### Embodiment Six

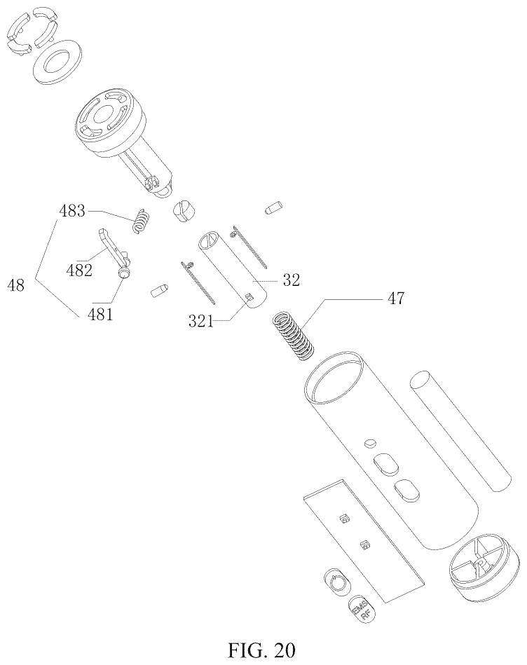

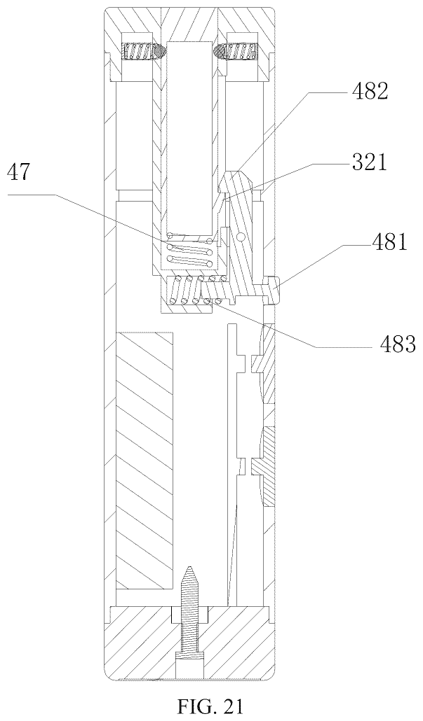

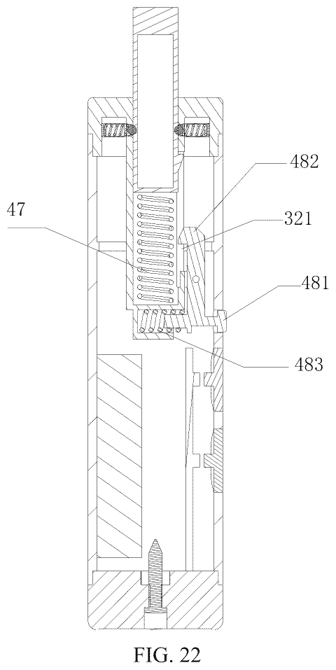

Referring to FIGS. 20 to 22, the difference between embodiment six and embodiment five lies in that the upward moving and downward moving of the second electrode assembly 3 depend on the operation of the user in embodiment five. In embodiment six, the upward moving process of the second electrode assembly 3 is changed to be automatic. Specifically, the lift driving member 4 includes an elastic member 47 and a release member 48. When the second electrode assembly 3 is located in the first position, the elastic member 47 retracts in the axial direction of the lifting rod 32 and is in an energy storage state. The release member 48 is configured to unlock the second electrode assembly 3 from the first position. In this case, the user can unlock the second electrode assembly 3 from the first position only through the release member 48. Then the elastic member 47 can release elastic reset force so that the second electrode assembly 3 is automatically driven to move upward.

To implement the preceding locking and unlocking functions, the release member 48 includes a button 481. The button 481 is connected to a swing rod 482. An engagement block 321 for engaging with and limiting a swing end of the swing rod 482 is disposed on the lifting rod 32. When the second electrode assembly 3 is located in the first position, the swing end engages with and limits the engagement block 321 so as to secure the second electrode assembly 3 in the first position. When the button 481 is pressed, the engagement block 321 disengages from the swing end and stops limiting the swing end.

Additionally, the button 481 is further connected to a reset spring 483. When the button 481 is pressed, the reset spring 483 accumulates the elastic reset force. In this case, when the user releases the button 481, the button 481 can be automatically reset to the initial position.

The above are only preferred embodiments of the present disclosure and are not intended to limit the design concept of the present invention. Any insubstantial changes, made by those skilled in the art, of the present disclosure within the technical scope of the present disclosure belong to an act of infringing the scope of the present disclosure.

1. A beauty apparatus, comprising a housing, an electrode assembly, and a lift driving member;
wherein the electrode assembly comprises a first electrode assembly and a second electrode assembly, and the lift driving member is located in the housing and configured to drive the second electrode assembly to move between a first position and a second position; and
when the second electrode assembly is located in the first position, an effective area of the electrode assembly capable of acting on a human skin surface is S1; and when the second electrode assembly is located in the second position, an effective area of the electrode assembly capable of acting on the human skin surface is S2; wherein S1>S2.

2. The beauty apparatus according to claim 1, wherein the second electrode assembly comprises a second electrode tip and a lifting rod, the lifting rod is securely connected to the second electrode tip, and conductive pieces are respectively disposed on two sides of the lifting rod in a radial direction of the lifting rod; and
the first electrode assembly is provided with a mount for mounting a first electrode tip, the mount is provided with an avoidance cavity for accommodating the second electrode assembly, and an inner wall of the avoidance cavity has contact points respectively electrically connected to the conductive pieces.

3. The beauty apparatus according to claim 2, wherein the lift driving member drives, based on manpower or electricity, the lifting rod to move in an axial direction of the lifting rod.

4. The beauty apparatus according to claim 3, wherein the lift driving member comprises a gear motor and a rotation sleeve, the gear motor is configured to drive the rotation sleeve to rotate, the rotation sleeve is connected to the lifting rod through a conversion member, and the conversion member is configured to convert a rotation of the rotation sleeve into a movement of the lifting rod in the axial direction of the lifting rod.

5. The beauty apparatus according to claim 3, wherein the lift driving member comprises a rotary knob and a rotation sleeve, the rotary knob is configured to drive the rotation sleeve to rotate, the rotation sleeve is connected to the lifting rod through a conversion member, and the conversion member is configured to convert a rotation of the rotation sleeve into a movement of the lifting rod in the axial direction of the lifting rod.

6. The beauty apparatus according to claim 5, wherein the rotary knob is disposed on a bottom of the housing in an axial direction of the housing, and the rotary knob is securely connected to the rotation sleeve through a connection rod.

7. The beauty apparatus according to claim 5, wherein the rotary knob is disposed on a top of the housing in an axial direction of the housing, and the rotary knob is secured to the rotation sleeve by inserting.

8. The beauty apparatus according to claim 7, wherein the rotary knob is the mount.

9. The beauty apparatus according to claim 4, wherein the rotation sleeve is provided with an avoidance channel for the lifting rod to pass through; the conversion member comprises a spiral guide rail and a guide block, wherein the spiral guide rail is disposed on an inner wall of the avoidance channel, and the guide block is disposed on the lifting rod; and the guide block is disposed in the spiral guide rail.

10. The beauty apparatus according to claim 3, wherein the lift driving member comprises a push rod motor, and a telescopic end of the push rod motor is cooperatively linked to the lifting rod.

11. The beauty apparatus according to claim 3, wherein the lift driving member comprises a push button securely connected to the lifting rod, the housing is provided with a sliding recess for the push button to translate, the sliding recess extends in the axial direction of the lifting rod, one end of the push button is securely connected to the lifting end, and another end of the push button is exposed outside the sliding recess to form an operation end.

12. The beauty apparatus according to claim 3, wherein the lift driving member comprises an elastic member and a release member; when the second electrode assembly is located in the first position, the elastic member retracts in the axial direction of the lifting rod and is in an energy storage state; and the release member is configured to unlock the second electrode assembly from the first position.

13. The beauty apparatus according to claim 12, wherein the release member comprises a button; the button is connected to a swing rod; an engagement block for engaging with and limiting a swing end of the swing rod is disposed on the lifting rod; when the second electrode assembly is located in the first position, the swing end engages with and limits the engagement block so as to secure the second electrode assembly in the first position; and when the button is pressed, the engagement block disengages from the swing end and stops limiting the swing end.

14. The beauty apparatus according to claim 1, wherein an electrode in the first electrode assembly and an electrode in the second electrode assembly are each a micro-current positive or negative contact point, or are each a radio frequency electrode.

15. The beauty apparatus according to claim 2, wherein in a process of the second electrode assembly moving from the first position to the second position, the second electrode tip in the second electrode assembly protrudes outward or recesses inward relative to the first electrode tip in the first electrode assembly.

16. The beauty apparatus according to claim 1, wherein the second electrode assembly and the first electrode assembly are arranged concentrically from inside to outside in a radial direction of the first electrode assembly and the second electrode assembly.

17. The beauty apparatus according to claim 5, wherein the rotation sleeve is provided with an avoidance channel for the lifting rod to pass through; the conversion member comprises a spiral guide rail and a guide block, wherein the spiral guide rail is disposed on an inner wall of the avoidance channel, and the guide block is disposed on the lifting rod; and the guide block is disposed in the spiral guide rail.

18. The beauty apparatus according to claim 6, wherein the rotation sleeve is provided with an avoidance channel for the lifting rod to pass through; the conversion member comprises a spiral guide rail and a guide block, wherein the spiral guide rail is disposed on an inner wall of the avoidance channel, and the guide block is disposed on the lifting rod; and the guide block is disposed in the spiral guide rail.

19. The beauty apparatus according to claim 7, wherein the rotation sleeve is provided with an avoidance channel for the lifting rod to pass through; the conversion member comprises a spiral guide rail and a guide block, wherein the spiral guide rail is disposed on an inner wall of the avoidance channel, and the guide block is disposed on the lifting rod; and the guide block is disposed in the spiral guide rail.

20. The beauty apparatus according to claim 8, wherein the rotation sleeve is provided with an avoidance channel for the lifting rod to pass through; the conversion member comprises a spiral guide rail and a guide block, wherein the spiral guide rail is disposed on an inner wall of the avoidance channel, and the guide block is disposed on the lifting rod; and the guide block is disposed in the spiral guide rail.
